(19) United States
(12) Patent Application Publication
(10) Pub. No.: US 2002/0169452 A1
(43) Pub. Date: Nov. 14, 2002

(54) Minimally traumatic surgical device for tissue treatment

(76) Inventors: Pertti Tormala, Tampere (FI); Tero Valimaa, Tampere (FI); Kimmo Lahteenkorva, Tampere (FI)

Correspondence Address: KENYON & KENYON, 1500 K STREET, N.W., SUITE 700, WASHINGTON, DC, 20005, US

(21) Appl. No.: 09/853,760

(22) Filed: May 14, 2001

(51) Int. Cl. A61B 17/84

(52) U.S. Cl. 606/72

(57) ABSTRACT

A fastener for repairing tissue or for attaching generally fibrous matter to tissue comprising a shaft comprising a curved, tapered proximal end configured to arrest the movement of the fastener in the distal direction, and a distal portion having protrusions for allowing movement in the distal direction and arresting movement in the proximal direction.

### FIELD OF THE INVENTION

The present invention relates to surgical implants for repairing tissue or attaching matter to tissue. More specifically, the present invention relates to a surgical fastener or device (implant) formed in the shape of an arrow comprising a shaft with a proximal (first) portion having a curved, tapered end and a distal portion having protrusions and, preferably, a pointed end. The tapered, curved form of the proximal portion and the protrusions of the distal portion lock the implant in a position mostly inside and partially on a soft and/or tough tissue or totally inside of soft and/or tough tissue. Further, the implant of the present invention may be used to affix another implant, like a hernia mesh, to or in a tissue. The implant may be manufactured of a polymer or a polymeric compound which is substantially (bio)absorbable in tissue conditions and contains oriented reinforcing structure or the like of a polymer or polymeric compound or ceramic compound, such as bioactive glass or tricalcium phosphate.

The surgical implant of the invention is particularly but not solely intended to be used in surgery to repair traumas to soft and/or tough tissues containing fibrous structures. More specific applications of the implant of the present invention could include, for example, the repair of ruptures of the meniscus of the knee, closing wounds to connective tissues, affixing synthetic hernia meshes or nonwoven collagen felts to tissue.

### BACKGROUND OF THE INVENTION

It has been shown that the fixation of meniscus traumas, like ruptures and lesions, by suturing with absorbable sutures gives better results than the removal of traumatized meniscal tissue, see e.g. N. A Palmeri, T. F. Winters, A. E. Joiner and T. Evans, “The Development and Testing of the Arthroscopic Meniscal Staple”, Arthroscopy, Vol. 5, No. 2, 1989, p. 156. However, arthroscopic suturing is a complicated and tedious technique where risks for the patient are significant because of the danger of damaging vessels and nerves. Therefore, for a long time surgeons have desired an absorbable meniscus lesion fixation device, like a staple or fastener, which has the advantages of absorbable suturing techniques but which can be used more rapidly and safely than sutures.

Several research groups have tried to develop absorbable meniscus lesion fixation devices such as clamps. However, the various demands upon such a device are high. It must be strong enough to maintain good contact of lesion tissues after the operation so that rapid healing occurs. The device must also retain its strength long enough to allow for good healing. It must be absorbed without causing complications that would prevent or hinder the healing of the lesion. Additionally, the installation of the device should be easy and rapid and should cause minimum operational trauma. Because of these high demands, the optimal absorbable meniscus lesion fixation device has not yet been developed. Palmeri et al. reported the development of a method of meniscal repair using arthroscopically applied absorbable fasteners. However, the reported method was complicated because the final design used cannulation of the staple for needle-guided placement. Additionally, staple fracture, migration and articular abrasion was found.

U.S. Pat. No. 4,873,976 discloses an arrow-like implant particularly intended for the surgical repair of meniscal ruptures. However, the arrow-like implant according to this publication has the disadvantage that particularly its proximal end (stem) may cause tissue irritation and abrasion, particularly when placed in connection with the meniscus because the stem may be left protruding from the outer surface of the meniscus.

Bays et al. (U.S. Pat. Nos. 4,884,572 and 4,895,141) describe a surgical-repair tack and applicator and method of using them. The tack has a barb member, a shaft portion and a grip portion. The tack is made of biodegradable material having a degradation time selected to coincide with the healing time of the tissue. In an alternate embodiment, the tacks's barb comprises a continuous helical barb. A disadvantage of this tack is that the grip portion is bulky and may remain on meniscal surface causing irritation inside a joint cavity.

The method and apparatus for repairing a meniscal tear disclosed by Winters (U.S. Pat. No. 5,059,206) comprises a fastener having protrusions or barbs that is applied to a meniscal tear with a delivery device. The delivery device has a flexible tip that is manipulable through a curved radius to enable the surgeon to insert the device into the central part of the knee and then extend the fastener radially outward into and across a meniscal tear. Also in this case the proximal end of the fastener is bulky comprising a cylindrical end (head member) which protrudes partially above and/or below the outer surface of the meniscus.

Tamminmäki et al. (U.S. Pat. No. 5,562,704) disclose an arrow-like bioabsorbable implant particularly intended for the surgical repair of meniscal ruptures. This implant does not have the guiding or abrasion problems that implants of U.S. Pat. No 4,873,976 or U.S. Pat. No. 5,059,206 may have. However, a disadvantage of U.S. Pat. No. 5,562,704 is that the proximal part of the implant (the wings) preferably remains on the surface of the meniscus, so that when the wings break as a consequence of bioabsorption, the broken wings may irritate knee joint tissues. If the proximal part with the wings is desired to be located inside of meniscal tissue, the surface capsule of the meniscus must be cut horizontally with a special cutting blade. This lengthens the operation time and may cause damage to the meniscus surface.

U.S. Pat. No. 5,569,252 describes a fastener, an installation device, and method for repairing tears in the soft tissue of a patient, including meniscal tears. The fastener has a variable-pitch helical protrusion along a central portion that decreases from the distal end to the proximal end, which can serve to bring two sides of the tear into opposition as the fastener is advanced across the two sides of the tear in a screwing motion. This implant, which needs a screwing motion for installation, is slow and tedious to use arthroscopically and the turning of the implant through fibrous tissue, such as meniscus tissue, has the risk that the fibrous tissue may twist around the turning implant, hindering or preventing the installation of the implant, or damaging the tissue.

Orthopedic and Musculoskeletal Markets Biotechnology and Tissue Engineering, Medical Data International, Inc., Irvine, Calif., USA, February 1997, p. 1-17 describes a bioabsorbable device for meniscal repair. This device has two legs with molded barbs that are attached by a flexible member composed of resorbable suture. The device is installed into a meniscus with an arthroscopic tool so that the legs penetrate the rupture of meniscus to hold the edges together. However, the two-leg device requires a bulky installation tool which makes arthroscopic installation of the device difficult.

U.S. patent application Ser. No. 08/887,130 describes a fastener for body tissue repair comprising: a shaft comprised of a proximal portion, having an upper surface and a lower surface with first protrusions, and a distal portion, said distal portion having a sharpened tip and one or more first protrusions, wherein said first protrusions have proximal surfaces configured to arrest the movement of the shaft in the proximal direction and distal surfaces configured to permit the movement of the shaft in the distal direction, said proximal portion having second protrusions on the upper surface and lower surface of the proximal portion, wherein said second protrusions have distal surfaces configured to arrest the movement of the shaft in the distal direction. Although this implant sinks totally inside a tissue, like knee meniscus, the second protrusions can be damaged, bent or broken during the insertion of the implant into tissue.

### SUMMARY OF THE INVENTION

It is an object of the present invention to provide a bioabsorbable fastener that allows a minimally invasive method for repairing a tear in soft or tough tissue and/or for fixation of synthetic fibrous implants or living tissue transplants on or in a living tissue.

It is a further object to provide a fastener that is rapid and easy to install and gives a strong and safe fixation of the tissue tear, implant or transplant, and is minimally traumatic. The fastener may be made from a nontoxic, biocompatible bioabsorbable polymer, polymer alloy or fiber reinforced polymer composite, specially designed to maintain its structural integrity during the healing of the tear and to prevent tissue abrasion.

It is an additional object to provide such a fastener having a shape designed to compress the tear.

It is a further object to provide a device (fastener) that, can penetrate the tissue being repaired (such as a meniscal tear) and hold the ruptured edges together while causing a minimal trauma to the tissue through which the fastener travels.

It is a further object to provide a device (fastener) that, once installed, will leave only a small part of the proximal end of the fastener on the surface of the tissue.

These and other objects may be attained with the fastener of the present invention.

The fastener of the present invention is designed for repairing a tear in soft and/or tough tissue of a patient, such as a tear of the meniscus within the knee.

The surgical fastener or device (implant) of the present invention has been formed in the shape of an arrow comprising a shaft with a proximal (first) portion having a tapered, curved end and a distal portion having protrusions and, preferably, a pointed end. The tapered, curved form of the proximal portion and the protrusions of the distal portion lock the implant in a position totally inside of soft and/or tough tissue or mostly inside and partially on a soft and/or tough tissue.

In the preferred embodiments, protrusions emerge from the distal portion of the device. The protrusions are typically barbs, scales, threads, serrations, ridges or the like. These protrusions at the distal portion of the shaft of the device help to prevent the installed device from slipping out of the tissue in the direction opposite to the direction of installation. At least one or more of the protrusions must penetrate the rupture plane inside of tissue to lock the distal portion of the device into the tissue distally of the tear. The tapered, curved, sharp form of the proximal portion of the shaft allows the locking of the shaft into the meniscal tissue when the fastener is pushed, shot or hammered into the tissue. Because of the curved, tapered structure, the location of the curved tip of the proximal portion of the shaft on the meniscal tissue causes no or only minimal trauma, and disturbance to the opposite tissue above it. The fastener may be buried totally into the meniscal tissue or leave only a small part of the fastener on the tissue surface. Generally, during and after installation, the forward movement of the fastener stops when the tapered, curved proximal tip of the device grabs the meniscal surface (and preferably penetrates a bit into the meniscal tissue). The tapered, curved, sharp proximal portion prevents the further penetration of the fastener into meniscal tissue because its sharp tip is locked into the meniscal surface. In this way the combined effect of distal protrusions and proximal tapered, curved portion lock the fastener effectively in relation to the meniscus to close and fixate the meniscal rupture to enhance its healing. However, it is possible to force the certain embodiments of fastener also totally inside of meniscus by force using a long piston of an installation cannula.

In a preferred embodiment, the protrusions of the distal portion of the shaft of the device are formed so that they facilitate the slipping of the device into the meniscus during insertion but they resist the movement of the device in the direction opposite to the installation direction. On the other hand, the tapered, curved, sharp proximal portion of the shaft of the device is formed so that it stops the forward movement of the device when it has been pushed or shot into the meniscus with the delivery (installation) tool. Both distal protrusions and proximal tapered, curved portion, acting together, exert an advantageous compression to the ruptured surface when the device is shot into the meniscus and across the rupture. This compression serves to close the rupture and promotes healing.

A further advantageous feature of the device is that the surface of the shaft may include longitudinal ridges. The ridges promote healing of the rupture by providing channels along the interiors of the ridges through which beneficial blood flow can occur along the length of the device. These channels, which are typically about 0.05-0.5 mm wide, act as capillaries, transporting blood from the highly vascularized distal portion of the meniscus to the poorly vascularized proximal portion of the meniscus. Further, the ridges help to guide the fastener through the cannula of the installation instrument and into the meniscal or other soft tissue during installation.

### BRIEF DESCRIPTION OF THE DRAWINGS

FIGS. 1A-1G illustrate, in a perspective view, various embodiments of fasteners in accordance with the present invention.

FIGS. 2A-2D illustrate, as viewed from the proximal end of the fastener, various embodiments of fasteners in accordance with the present invention.

FIG. 3 illustrates, in a perspective view, an embodiment of a ridged fastener in accordance with the present invention.

FIGS. 4-7 illustrate, in cross section, various embodiments of fasteners in accordance with the present invention.

FIGS. 8A-D illustrate as a cross-section the installation of a fastener of the present invention into a torn meniscus.

FIGS. 9-10 illustrate the fibrous structures of the meniscus of the knee.

FIGS. 11A-B illustrate the orientation of the fibrous structure of the meniscus in relation to installed fasteners of the invention.

FIG. 12 illustrates, as seen from proximal direction, the location of the proximal, tapered curved tip of the fastener of FIGS. 1A, 2A and 11A, on the surface of a meniscus.

FIGS. 13A-B illustrate the fixation of a fibrous mesh on the surface of living tissue by means of fasteners of this invention.

### DETAILED DESCRIPTION OF THE PREFERRED EMBODIMENTS

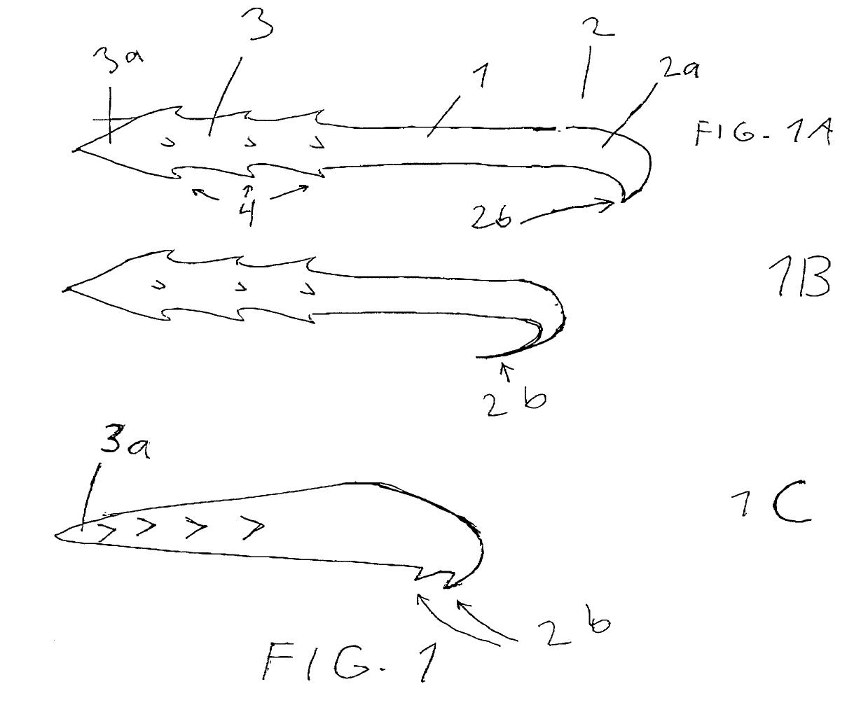

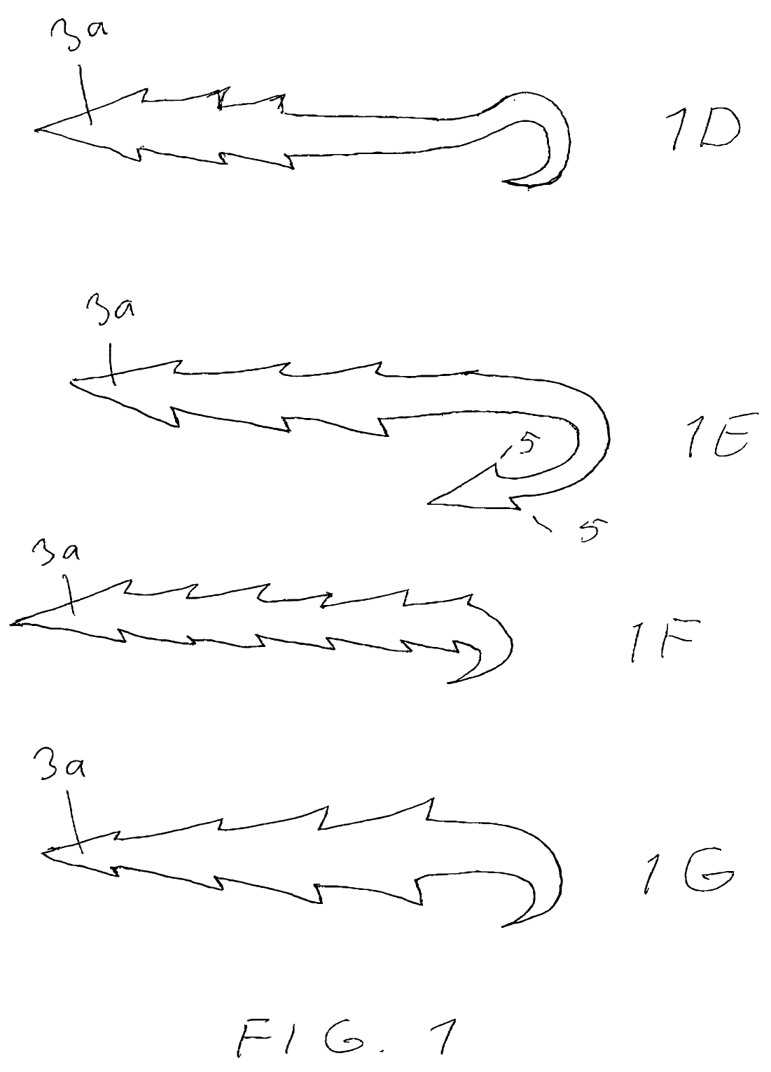

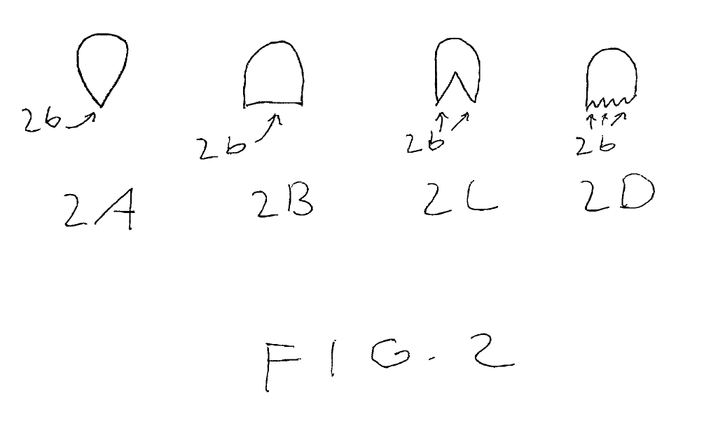

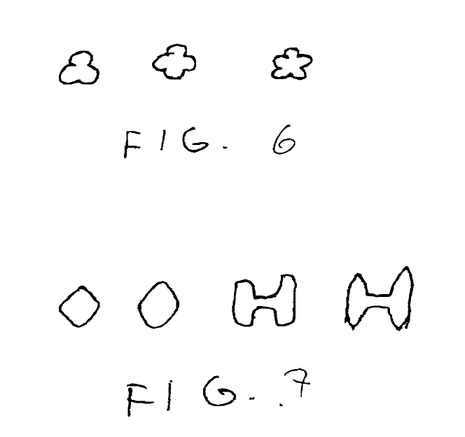

A description of the preferred embodiments of the present invention is presented with reference to FIGS. 1-12.

A preferred exemplary embodiment of the present invention comprises a fastener and method for repairing a soft or tough, tissue, such as the meniscus of the knee, in a human patient.

FIGS. 1A-1G illustrate, as viewed from the side, some preferred embodiments of the fastener of the present invention. It is designed to have an arrow shape, and it comprises a shaft 1, whose proximal portion 2 is formed with a tapered, curved end 2a with a sharp tip 2b for the purpose of providing the locking element to lock the fastener 1 in relation to the meniscus with the proximal part 2 remaining at least partially on the surface of the meniscus, and whose distal portion 3 is formed with a head that in a preferred embodiment has a sharp tip 3a. The distal portion comprises distal protrusions 4 with respect to the shaft 1. The distal protrusions 4 in FIGS. 1A-1G are small, sharp-profile barbs. The protrusions 4 can also take the form of, e.g., ridges, threads, scales, or pyramids. When the fastener of the present invention is used, for instance, to repair tears of the meniscus, the purpose of the curved proximal end portion 2a is to stop forward movement of the implant. During the final stage of the installation of the implant of the present invention, the proximal tapered, curved end 2a will sink totally inside of the meniscus or remain partially on the surface of meniscus. The tapered tip 2b hooks itself inside of the meniscus or on the meniscal surface and stops the device and prevents its further sinking into the meniscus. Using a proper pushing device and force, the fastener may be forced totally inside of the meniscus or at least deep enough into the meniscus that the tapered, curved end 2a is located at the bottom of a small notch on the surface of the meniscus, thus causing no disturbance to the opposite joint cartilage surface of the distal joint surface of the femur.

The tip 2b of the fasteners of the present invention may have different geometries. The fastener of FIG. 1A has a relatively short tip 2b of curved end 2a, while FIG. 1B illustrates an embodiment with a longer, needle-like tip 2b. As seen in FIG. 1C, the fastener of the present invention may have more than one sharp tip 2b. FIG. 1D illustrates yet another preferred embodiment of the fastener of the present invention. FIG. 1E illustrates a fastener, where the proximal end of the fastener is equipped with two additional protrusions 5. As seen in FIG. 1F, a fastener of the present invention may have protrusions all along the length of its shaft. As seen in FIG. 1G, the shaft of the fastener of the present invention need not be of uniform length and can, for instance, have a slightly conical shape.

The tapered proximal portion 2a of the fasteners of the present invention also may have different geometries, some of which are illustrated in FIG. 2. In FIG. 2A, the proximal portion 2a is tapered to a point. In this embodiment the end 2a with a sharp tip 2b forms a fishhook like element which may penetrate into the meniscus surface for effective stopping of the end 2a on the surface of the meniscus during surgical installation procedure. In the embodiment of FIG. 2B, the proximal tip 2b of the curved end 2a is flat. Such a flat proximal tip remains on meniscal tissue causing only a minimal disturbance into the internal structure of the meniscus. However, such a flat and broad proximal tip 2b stops the fastener end 2a effectively on meniscal tissue. In the embodiment of FIG. 2C, the proximal tip 2b is pronged, like an end of a crowbar, enhancing the locking effect on meniscal tissue. FIGS. 2D shows an embodiment with a proximal end 2a having several small tips 2b.

Further, both the tip 2b and tip 3a can have different cross-sectional geometries. They can be, e.g., circular, triangular, or squared, etc.

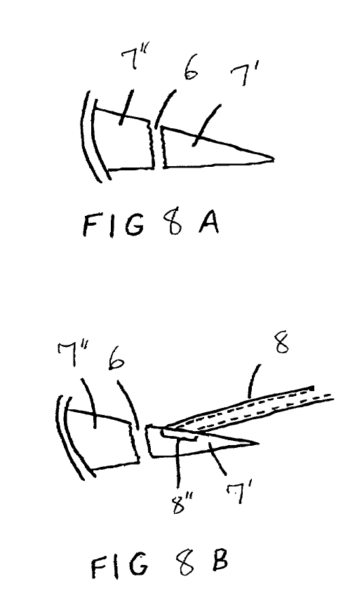

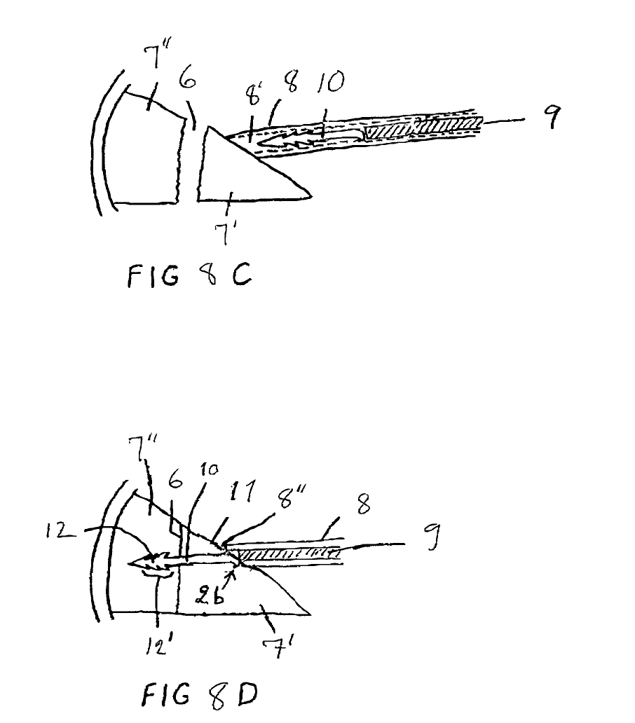

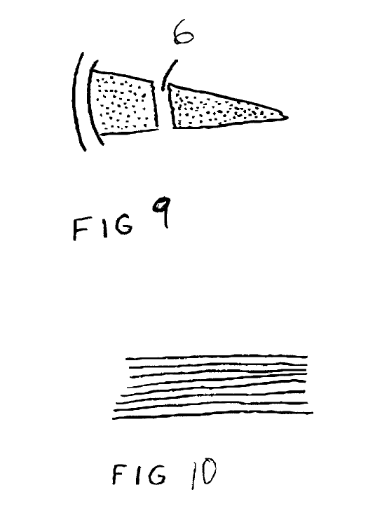

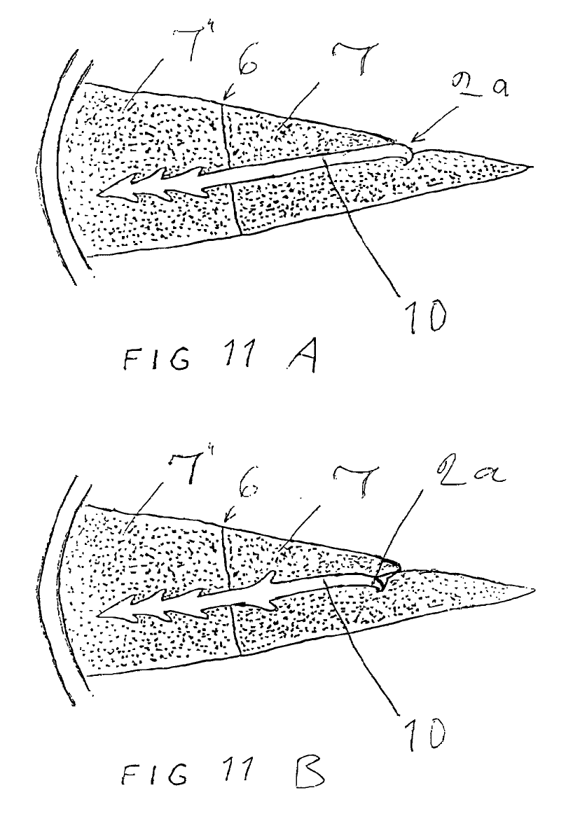

The proximal tapered portion 2a with curved tip(s) 2b and distal protrusions 4 effectively lock the device totally or partially inside of the meniscus preventing its movement both in the direction of installation and in the direction opposite to it. Additionally, the installation compresses the rupture surface (see FIGS. 8A-D) because the proximal tapered, curved portion 2a with tip 2b pushes the meniscus, and therefore also the proximal side of the rupture against the distal side of the rupture (see FIGS. 8A-8D), during the final phase of installation.

Because the device is located mainly inside of the meniscus, leaving only a small prominence on the meniscus surface, the risks of prior art devices, regarding the complications originating (a) from the presence of the bulky proximal part of the device on the meniscal surface, or (b) from the cutting of collagen fibers inside of meniscus by the first (proximal) protrusions, are greatly reduced or eliminated.

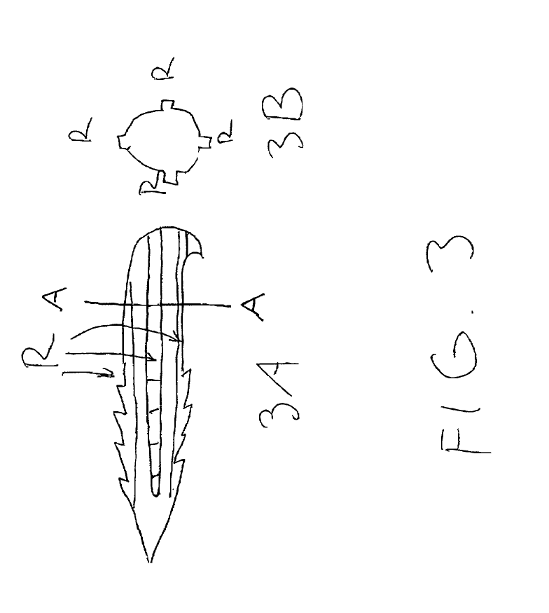

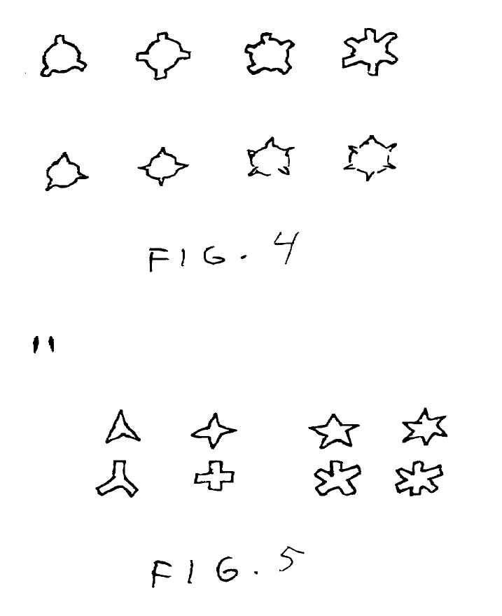

In a further preferred embodiment, the surface of the fastener can also include longitudinal ridges, into which the arresting means (proximal tapered, curved portion and distal protrusions) can be machined or molded. FIG. 3A shows a side-view perspective of such a fastener having on its surface longitudinal ridges (R), which are arranged onto the surface of the fastener as shown in FIG. 3B, which illustrates the cross-section of the fastener in the plane A-A of FIG. 3A.

The distal protrusions (such as barbs) can be machined effectively into the longitudinal ridges.

Other types of distal protrusions, than those described in FIG. 1 and 3, can be used in the fasteners of the invention. Such protrusions are described e.g. in U.S. patent application Ser. No. 08/887,130, which is hereby incorporated by reference in its entirety.

There are numerous possible arrangements for the longitudinal ridges on the surface of the fastener. Also the geometry of the ridges can be varied to influence the gripping capacity of the protrusions and of the tapered proximal end on meniscal or other soft tissue. FIGS. 4-7 illustrate some preferred embodiments of the cross-sectional structures of ridged fasteners of the present invention.

FIGS. 8A-D illustrate a preferred method for installing fasteners of the invention into ruptured meniscal tissue. FIG. 8A illustrates as viewed from the side, a meniscus with a rupture, 6, separating the meniscus into a proximal side, 7′, and a distal side, 7″. As seen in FIG. 8B during the operation the tip, 8″ of an installation cannula 8 is pushed into the knee joint through a small incision and the tip is located on the surface of the proximal part of the meniscus 7′ (in relation to the rupture 6).

As seen in FIG. 8C, piston 9 moves to the left (distally) and pushes the fastener 10 through the hole 8′ inside of cannula 8. The piston 9 can be accelerated to a high speed so that the piston 9 pushes (shoots) the fastener 10 with a high speed into the meniscus as is illustrated in FIG. 8D. The piston 9 stops at the final stage of its movement (by way of, e.g., a stopper (not shown) at the proximal end of the piston 9), typically so that the tip of the piston 9 protrudes out of the tip 8″ of cannula 8 about 0.5-1 mm. This pushes the fastener inside of the meniscal tissue so that the proximal end 2a of the fastener is located at the bottom of a small notch formed on the surface of the meniscus. When the location of the cannula tip 8″ on the meniscal surface is selected in a proper way, typically 2-4 mm in front of the meniscal tear 6, and the direction of the cannula is proper, the fastener penetrates the proximal meniscus part 7′, the tear plane 6 and closes the tear with the compression force created with the installation push. As seen in FIG. 8D, the piston 9 pushes and forces the fastener 10 inside of the meniscal tissue so that the tip 2b of the proximal end 2a penetrates into the surface of meniscus and the end 2a is left partially on meniscal surface into a small notch. When the tapered proximal end 2a of the fastener is forced onto the meniscal tissue (see FIG. 8D), it pushes into the proximal part of meniscus 7′, closing the rupture 6. As soon as the piston 9 stops (typically 0.5-1 mm below the surface of the meniscus) the proximal tapered portion 2a with the tip 2b stops the fastener and prevents its further movement into meniscal tissue. On the other hand, the distal portion of the device 12 is pushed across the rupture 6 and into the distal side of the meniscus 7″, where the distal protrusions 12′ prevent the slipping of the fastener back in the direction opposite to the installation direction. Accordingly, the rupture 6 is closed effectively, the fastener is locked in position to keep the rupture 6 closed, and only a small part of the whole fastener is left on the surface of the meniscal tissue.

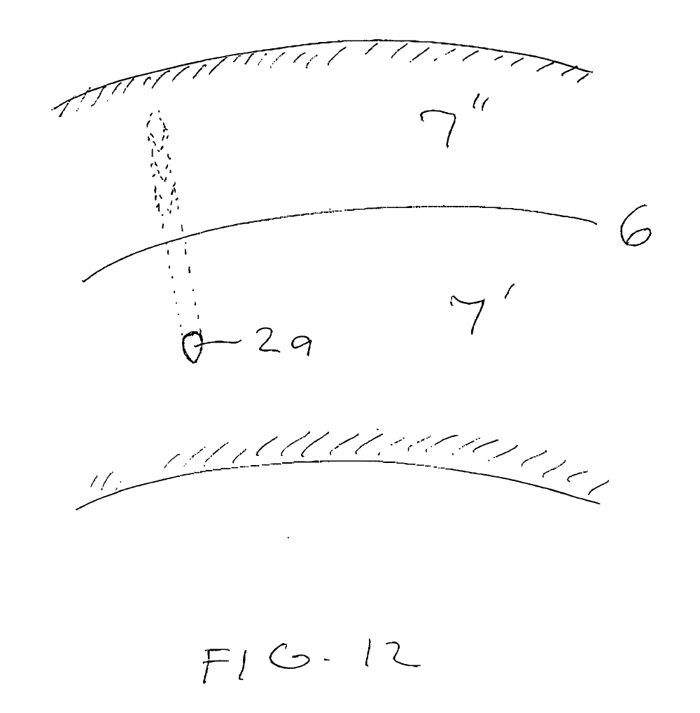

It is typical that the microstructure of a meniscus contains reinforcing collagen fibers. Inside of a meniscus, many collagen fibers are oriented in a horizontal plane nearly parallel to the lower surface of the meniscus. If the horizontal collagen fibers are examined in a cut cross-section of a meniscus (as shown in FIGS. 8A-D) their cut ends can be seen microscopically as points on the cross-sectional surface, as represented in FIG. 9. The typical vertical meniscus lesion (rupture) 6 develops between the long axes of collagen fibers, because the binding forces between collagen fibers are weaker than along the long axis of fibers. If the internal collagen fiber structure of a meniscus is examined from the direction of the long axis of the fastener, i.e., from the direction from which the fastener enters the meniscus, the collagen fibers are seen as parallel, horizontal fiber bundles, as is shown schematically in FIG. 10.

Because of the special arrangement of most of the reinforcing horizontal collagen fibers inside of the meniscus, shown schematically in FIGS. 9 and 10, the distal protrusions and tapered, curved proximal end 2a and its tip 2b should be located at least on the upper and/or lower surface of the fastener, so that as the fastener penetrates into the meniscal tissue, the distal protrusions slide forward through the collagen fiber bundles and grab finally between the horizontal collagen fiber bundles, locking the fastener in place. On the other hand, it is advantageous that the proximal, curved portion is tapered into the direction of its proximal end so that in the most efficient case the sharp tip 2b penetrates between horizontal collagen fiber bundles and thus locks the proximal portion to its place when the installation is complete. This is shown schematically in FIG. 11A as a meniscal cross-section. According to another advantageous embodiment, the piston 9 of the installation cannula 8 (see FIGS. 8C-D) can be so long that it pushes the fastener by force totally inside of the meniscus. For instance, if the piston 9 is 3-4 mm longer than the cannula, it can push the fastener 10 totally inside of the meniscus, as is seen schematically in FIG. 11B. When the pushing force of piston has been released, the fastener will stop inside of the meniscus, because the curved, tapered proximal part 2a of the fastener prevents its slippage further into the distal direction.

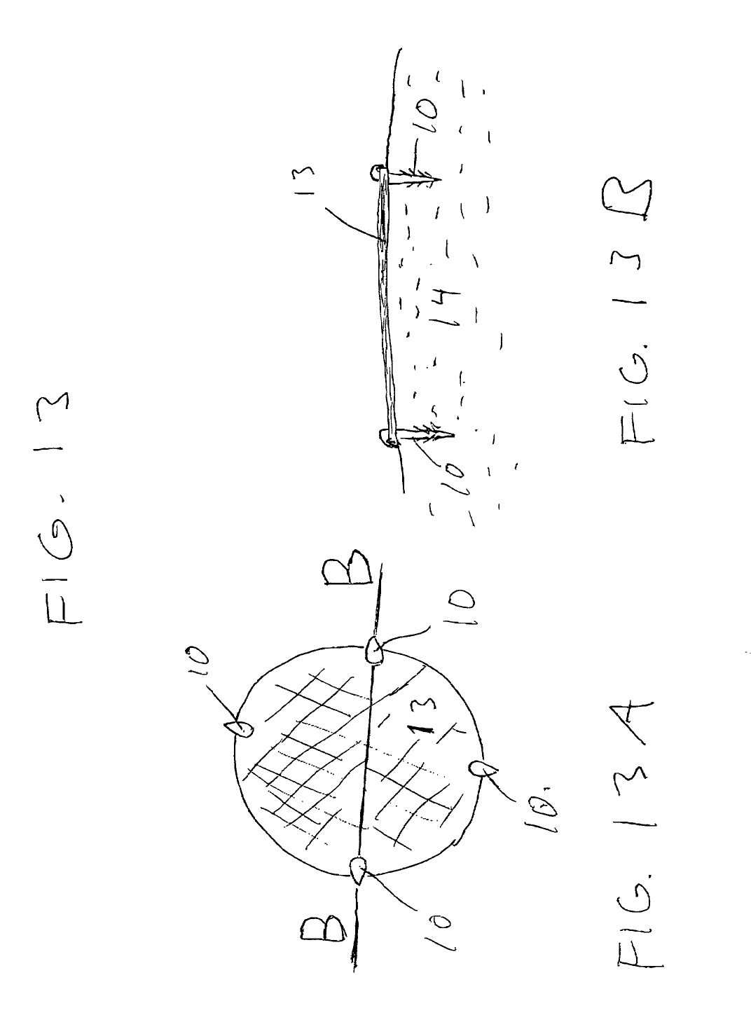

FIG. 12 describes the arrangement of FIG. 11A as seen from proximal direction on the surface of the meniscus. Only a small end 2a of the fastener is seen on the surface of the proximal side 7′ of meniscus. In an advantageous embodiment, the proximal end 2a of fastener 10 is located at the bottom of a small notch on the meniscal surface. 49. The meniscus also includes oriented fibers that are not horizontal. For example, the meniscus can also contain fibers having radial or oblique orientations. The collagen fibers form an essentially three-dimensional network in the meniscus, with such fibers being of particular importance with regard to using the present invention for treating the typical vertical (bucket handle) tears that occur.

In addition to securing tears in living tissues, these fasteners can also be used to affix synthetic fibrous implants, like membranes, meshes, non-woven felts, fibrous scaffolds, etc. on or in living tissues. Such synthetic fibrous implants are described e.g. in EPO Pat. No. 0423155, U.S. Pat. No. 6,007,580 and PCT/EP 98/03030.

When using the fasteners of this invention to affix synthetic fibrous implant on or into living tissue, the fibrous implant is first aligned on the surface or inside of the living tissue. Thereafter, fasteners are pushed one after another through the synthetic implant so that the barbed distal part of fastener locks the fastener into living tissue below the synthetic implant and the curved, tapered proximal end of the implant remains on the synthetic implant securing it to the surface (or inside) of the living tissue. FIG. 13A illustrates, as seen from above, and FIG. 13B illustrates, as a side view in plane B-B of FIG. 13A, how a fibrous mesh 13 may be secured with fasteners 10 on a living tissue 14.

The fasteners of this invention may also be applied for the fixation of living tissue transplants, like autografts, allografts and xenografts, such as collagen membranes and felts, periosteum transplants or connective tissue transplants.

The bioabsorbable implants of this invention can be manufactured of bioabsorbable polymers, copolymers or polymer mixtures or alloys using melt molding methods known in the prior art. It is also possible to use the techniques of U.S. Pat. No. 4,743,257 to mold in a compression or injection mold absorbable fibers and binding polymer together to create a fiber-reinforced or especially a self-reinforced structure. The implants of this invention can be molded in a single compression molding cycle, or the protrusions can be machined on the surface of a fastener after the molding cycle.

The oriented and/or self-reinforced structure can also be created during extrusion or injection molding of absorbable polymeric melt trough a suitable die or into a suitable mold at high speed and pressure. When cooling occurs at suitable conditions, the flow orientation of the melt remains in the solid material as an oriented or self-reinforcing structure. In an advantageous embodiment, the mold can have the form of the implant, but it is also possible to manufacture the implants of the invention by machining (possibly using heat) and thermoforming (e.g. by bending the proximal end) of injection-molded or extruded semifinished products.

It is also advantageous to make the implants of melt-molded, solid state drawn or compressed, bioabsorbable polymeric materials, which are described e.g. in U.S. Pat. No. 4,968,317 or 4,898,186.

The reinforcing fibers of the implant can also be ceramic fibers, like bioabsorbable hydroxyapatite or bioactive glass or tricalcium phosphate fibers. Such bioabsorbable, ceramic fiber reinforced materials are described e.g. in European Patent Application No. 0146398 and in WO 96/21628.

Oriented and/or self-reinforced or otherwise fiber reinforced implants of this invention can be manufactured by molding the reinforcement fiber-polymer matrix to the final product in a mold, whose mold cavity has the form of the final product or the final form can be machined mechanically (possibly also using heat) on a preform, such as a melt-molded and solid-state drawn rod, as is described e.g. in U.S. Pat. No. 4,968,317.

In some advantageous embodiments of this invention, the orientation and/or reinforcing elements of the self-reinforced structure are mainly oriented in the direction of the long axis of the shaft of the implant and also into the tapered, curved proximal end. The reinforcement elements may extend into any protrusions or ridges of the implant, and also into the tapered, curved proximal end. The reinforcements elements can also turn spirally around the long axis of the implant and also into the tapered, curved proximal end. Also other different orientations of reinforcement elements in elongated samples which are familiar from composite technology can be applied to the present invention. However, a general feature of orientation and/or fiber-reinforcement or self-reinforcement of the implants of this invention is that many of the reinforcing elements are oriented in such a way that they can carry effectively the different external loads (such as tensile, bending and shear loads) that are directed to the healing rupture (for example, loads to a meniscus caused by the movement of the patient's knee).

According to another advantageous embodiment of the invention, the meniscal repair implant, or a special coating layer on its surface, may contain one or more bioactive substances, such as antibiotics, chemotherapeutic substances, angiogenic growth factors, substances accelerating the healing of the wound, growth hormones and the like. Such bioactive meniscal repair implants are especially advantageous in surgical use, because they chemically contribute to the healing of the lesion in addition to providing mechanical support.

The oriented and/or reinforced materials of the implants typically have initial tensile strengths of 100-2000 MPa, bending strengths of 100-600 MPa and shear strengths of 80-400 MPa. Additionally, they can be made stiff and tough or flexible. These mechanical properties are superior to those of non-reinforced absorbable polymers which typically show strengths between 40 and 100 MPa and may additionally be brittle.

The fasteners of the present invention may be manufactured to be relatively thin (e.g. with shaft diameters of 0.2-to 1.5 mm) which minimizes the size of the portion of the fastener, if any, that is located on the tissue surface.

The implants of the present invention may be sterilized by any of the well known sterilization techniques, depending on the type of material used in manufacture of the implant. Suitable sterilization techniques include heat or steam sterilization, radiation sterilization such as cobalt 60 irradiation or electron beams, ethylene oxide sterilization, and the like.

After the description above of the present invention and certain specific embodiments thereof, it will be readily apparent to those skilled in the art that many variations and modifications may be made to the present invention without departing from the spirit and scope thereof.

We claim:

1. A fastener for body tissue repair comprising:
a shaft comprising a proximal portion and a distal portion,
said proximal portion having a tapered, curved end configured to arrest the movement of said shaft in the distal direction,
said distal portion having one or more protrusions, wherein said protrusions have proximal surfaces configured to arrest the movement of the shaft in the proximal direction and distal surfaces configured to permit the movement of the shaft in the distal direction.

2. A fastener according to claim 1, wherein a portion of said shaft further comprises one or more longitudinal ridges.

3. A fastener according to claim 2, wherein said protrusions protrude from said one or more longitudinal ridges.

4. The fastener of claim 3, wherein said one or more ridges run the length of said shaft.

5. A fastener according to claim 1 wherein said fastener comprises a bioactive material.

6. The fastener of claim 1, wherein said tapered, curved end further comprises protuberances having proximal surfaces configured to arrest the movement of the shaft in the proximal direction.

7. A method for the repair of a rupture in a meniscus, comprising the steps of: providing a fastener comprising:
a shaft comprising a proximal portion and a distal portion,
said proximal portion having a tapered, curved end configured to arrest the movement of said shaft in the distal direction,
said distal portion having one or more protrusions, wherein said protrusions have proximal surfaces configured to arrest the movement of the shaft in the proximal direction and distal surfaces configured to permit the movement of the shaft in the distal direction; aligning said fastener on the surface of the meniscus; and
pushing said fastener into the meniscus until said fastener bridges the rupture and only said curved end of said fastener is positioned on the surface of the meniscus.

8. A method for the repair of a rupture in a meniscus, comprising the steps of: providing a fastener comprising:
a shaft comprising a proximal portion and a distal portion,
said proximal portion having a tapered, curved end configured to arrest the movement of said shaft in the distal direction,
said distal portion having one or more protrusions, wherein said protrusions have proximal surfaces configured to arrest the movement of the shaft in the proximal direction and distal surfaces configured to permit the movement of the shaft in the distal direction;
aligning the fastener of claim 1 on the surface of the meniscus; and
pushing the fastener into the meniscus until said fastener bridges the rupture and is embedded totally within the meniscus.

9. A method for the fixation of a fibrous implant or tissue transplant on a living tissue comprising the steps of:
aligning the fibrous implant or tissue transplant on the surface of the living tissue; providing a fastener comprising:
a shaft comprising a proximal portion and a distal portion,
said proximal portion having a tapered, curved end configured to hold a portion of said fibrous implant,
said distal portion having one or more protrusions, wherein said protrusions have proximal surfaces configured to arrest the movement of the shaft in the proximal direction and distal surfaces configured to permit the movement of the shaft in the distal direction;
and pushing said fastener through said implant or tissue transplant and into the living tissue until said tapered, curved end contacts said implant or tissue transplant.

10. A method according to claim 9, wherein the fibrous implant or transplant is selected from the group consisting of: synthetic, polymeric mesh or scaffold, collagenous mesh or scaffold, periosteum transplant and connective tissue transplant.

11. A fastener for body tissue repair comprising:
a shaft including a proximal portion and a distal portion;
wherein said proximal portion includes means for arresting said shaft's movement in the distal direction; and
wherein said distal portion includes means for arresting said shaft's movement in the distal direction.

12. The fastener of claim 11, wherein the proximal portion further includes means for arresting said shaft's movement in the proximal direction.
